(19) United States
(12) Patent Application Publication
(10) Pub. No.: US 2026/0023086 A1
(43) Pub. Date: Jan. 22, 2026

(54) PROTEIN AGGREGATION ASSAY AND METHODS OF USING THE SAME

(71) Applicant: Board of Supervisors of Louisiana State University and Agricultural and Mechanical College, Baton Rouge, LA (US)

(72) Inventors: Christopher KEVIL, Shreveport, LA (US); Gopi KOLLURU, Shreveport, LA (US); Benjamin ESNEAULT, Shreveport, LA (US); Xinggui SHEN, Shreveport, LA (US)

(21) Appl. No.: 18/998,000

(22) PCT Filed: Jul. 28, 2023
(86) PCT No.: PCT/US2023/029016

§ 371 Date: Jan. 23, 2025

Related U.S. Application Data
(60) Provisional application No. 63/392,935, filed on Jul. 28, 2022.

(51) Int. Cl. G01N 33/68; G01N 33/50

(52) U.S. Cl. CPC G01N 33/6896; G01N 33/5008; G01N 2333/4709; G01N 2500/04

(57) ABSTRACT

The present invention is directed to a protein aggregation assay, and methods of use thereof.

This application is an International Application which claims priority from U.S. provisional patent application No. 63/392,935, filed on Jul. 28, 2022, the entire contents of which are incorporated herein by reference.

For countries that permit incorporation by reference, all patents, patent applications and publications cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted being prior art.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under HL147665, GM121307, and HL149264 awarded by the National Institutes of Health. The government has certain rights in the invention.

### FIELD OF THE INVENTION

This invention is directed to a protein aggregation assay, and methods of use thereof.

### BACKGROUND OF THE INVENTION

β-amyloid is the fibrillar form of the 40-42 amino acid peptide that forms the amyloid plaques found in Alzheimer's Disease (AD) patients. β-amyloid is derived from the amyloid precursor protein (APP), a membrane glycoprotein that plays a role in nervous system function. The 1-42 and 1-40 β-amyloid fragments are the most dominant in individuals with AD. The 25-35 fragment is known to contribute to aggregation and cytotoxicity in the nervous system. Alzheimer's Disease (AD) is characterized by the deposition of amyloid plaques between neurons in the brain.

### SUMMARY OF THE INVENTION

An aspect of the invention is directed to an in vitro drug screening method. For example, the method comprises incubating, for a period of time, a reaction mixture comprising at least one β-amyloid fragment peptide, at least one polysulfide, and at least one test candidate, and detecting the presence or absence of β-amyloid fragment peptide aggregation, wherein the presence or absence of β-amyloid fragment peptide aggregation is indicative of whether the test candidate prevents or reduces β-amyloid fragment peptide aggregation.

In embodiments, the β-amyloid fragment peptide comprises 1-40, 1-42, 25-35, or any combination thereof.

In embodiments, the polysulfide comprises Na₂S, Na₂S₂, Na₂S₃, Na₂S₄, organic sulfane sulfurs, polythionates, or any combination thereof.

In embodiments, detecting comprises an immunoassay, mass spectrometry, an aggregation assay, ultracentrifugation, size-exclusion chromatography, gel electrophoresis, or dynamic light scattering measurements. For example, the immunoassay is an immunoblot.

In embodiments. the level of β-amyloid fragment peptide aggregation is compared to that of a control sample. For example, the control sample comprises a positive control, a negative control, or both.

In embodiments, in vitro drug screening method further comprises selecting the test candidate as a therapeutic agent if β-amyloid fragment peptide aggregation is prevented or reduced.

In embodiments, the reaction mixture is incubated in a reaction vessel. For example, the reaction vessel comprises a dish, a tube, or a multiwell plate.

In embodiments, the at least one test candidate comprises a drug library.

In embodiments, the at least one test candidate comprises a nitric oxide (NO) or a test candidate that comprises NO.

In embodiments, the method is a high-throughput method.

Aspects of the invention are also drawn towards an in vitro method of identifying or monitoring β-amyloid fragment peptide aggregation in a sample. For example. the method comprises detecting β-amyloid fragment peptide aggregation in a sample comprising at least one β-amyloid fragment peptide, at least one polysulfide, and at least one test candidate; and selecting the test candidate as a therapeutic agent if β-amyloid fragment peptide aggregation is prevented or reduced.

In embodiments, the β-amyloid fragment peptide, at least one polysulfide, and at least one test candidate are co-incubated for a period of time in a reaction vessel.

Aspects of the invention are further drawn to a kit. For example, the kit comprises at least one β-amyloid fragment peptide, at least one polysulfide, at least one reaction vessel, a reaction media, and instructions for use.

In embodiments, the kit further comprises at least one test candidate. For example. the at least one test candidate comprises a drug library.

In embodiments, the kit further comprises at least one control. For example, the at least one control comprises a positive control, a negative control, or both.

Other objects and advantages of this invention will become readily apparent from the ensuing description.

### BRIEF DESCRIPTION OF THE FIGURES

FIG. 1 provides a schematic showing the primary amino acid sequence of the 42 amino acid β-amyloid. Adapted from Chen et al. 2017.

FIG. 2 provides data showing the median and distribution densities of H2S metabolites in control (C) and Alzheimer's Disease related dementia (A) individuals. Adapted from Disbrow et al. 2021.

FIG. 3 provides a schematic showing the amyloidogenic and non-amyloidogenic pathways of human amyloid precursor protein (APP). Adapted from Chen et al. 2017.

FIG. 4 provides western blot analysis for β-amyloid fragments. Panel A provides western plot data for β-amyloid fragment 1-42. Panel B provides western plot data for β-amyloid fragment 1-40.

FIG. 5 provides mass spectrometry analysis for β-amyloid fragments. Panels A-B provides mass spectrometry data for β-amyloid fragment 1-42; Panels C-D provides mass spectrometry data for β-amyloid fragment 1-40; and Panels E-F provides mass spectrometry data for β-amyloid fragment 25-35.

### DETAILED DESCRIPTION OF THE INVENTION

### Abbreviations and Definitions

Detailed descriptions of one or more preferred embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general. the term “about” is used herein to modify a numerical value above and below the stated value by a variance of 20 percent up or down (higher or lower).

The term “in vivo” can refer to an event that takes place in a subject's body.

The term “in vitro” can refer to an event that takes places outside of a subject's body.

The term “ex vivo” can refer to outside a living subject. Examples of ex vivo cell populations include in vitro cell cultures and biological samples such as fluid or tissue samples from humans or animals. Such samples can be obtained by methods well known in the art. Exemplary biological fluid samples include blood, cerebrospinal fluid, urine, saliva. Exemplary tissue samples include tumors and biopsies thereof. In this context, the compounds can be in numerous applications, both therapeutic and experimental.

Aspects of the invention are drawn to an in vitro drug-screening method for detecting the presence or absence of ß-amyloid fragment peptide aggregation.

The phrase “drug-screening method” can refer to a method that allows for high throughput screening of compounds at different time points. The screening process can allow for a deeper understanding of cell pathways that can be disrupted and/or affected by a treatment.

In embodiments, the method comprises incubating a reaction mixture comprising at least one β-amyloid fragment peptide, at least one polysulfide, and, optionally, at least one test candidate, and detecting the level of β-amyloid fragment peptide aggregation.

The term “reaction mixture” can refer to a mixture of components necessary to effect a reaction. In embodiments, the reaction mixture can comprise at least one β-amyloid fragment peptide, at least one polysulfide, and at least one test candidate. The reaction mixture can further comprise a buffer (e.g., a zwitterionic buffer).

A “β-amyloid fragment peptide” can refer to the fibrillar form of the 40-42 peptide produced through the proteolytic processing of a transmembrane protein, amyloid precursor protein (APP), by β- and γ-secretases. β-amyloid accumulation in the brain is proposed to be an early toxic event in the pathogenesis of Alzheimer's disease, which is the most common form of dementia associated with plaques and tangles in the brain. For example, the β-amyloid fragment peptide can comprise fragment 1-40, fragment 1-42, or fragment 25-35, or any combination thereof. β-amyloid fragment 1-40, sequence DAEFRHDSGYEVHHQKLVFFAEDVGSNKGAIIGLMVGGVV, is a peptide found in plaques in the brains of patients with Alzheimer's disease, and is shown to have both neurotrophic and neurotoxic effects. β-amyloid fragment 1-42, sequence DAEFRHDSGYEVHHQKLVFFAEDVGSNKGAIIGLMVGGVVIA, is a major component of amyloid plaques and accumulates in neurons of Alzheimer's disease brains. Biochemical analysis of the amyloid peptides isolated from Alzheimer's disease brain indicates that β-amyloid fragment 1-42 is the principal species associated with senile plaque amyloids. β-amyloid fragment 25-35, sequence GSNKGAIIGLM, is the shortest fragment that exhibits large β-sheet fibrils and retains the toxicity of the full-length peptide.

The term “aggregation” can refer to the tendency of a molecule or colloidal body to associate together into a mass or body of units or parts.

“β-amyloid fragment peptide aggregation” can refer to the aggregation of β-amyloids in the brain. At low concentrations, β-amyloids remain monomeric and function as antioxidants (Kontush, A., 2001). At higher concentrations, β-amyloids start aggregating—initially into small permeable oligomers that travel freely into the brain, and, in the later stages, into plaques that are hallmarks of Alzheimer's disease (Harper et al., 1999; Lomakin et al., 1997; Hardy and Selkoe, 2002).

The term “amino acid” can refer to naturally occurring and synthetic amino acids, as well as amino acid analogs and amino acid mimetics that operate in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that operates in a manner similar to a naturally occurring amino acid. Amino acids can be referred to herein by their known three letter symbols or by the one-letter symbols recommended by the IUPAC-IUB Biochemical Nomenclature Commission. Nucleotides, likewise, can be referred to by their accepted single-letter codes.

A “protein” can refer to any of a class of nitrogenous organic compounds that comprise large molecules composed of one or more long chains of amino acids and are an essential part of living organisms. A protein can contain various modifications to the amino acid structure such as disulfide bond formation, phosphorylations and glycosylations. A linear chain of amino acid residues can be called a “polypeptide.” A protein contains at least one polypeptide. Short polypeptides, e.g., containing less than 20-30 residues, are sometimes referred to as “peptides.”

The term “peptide” can refer to a polymer of amino acid residues ranging in length from 2 to about 30, or to about 40, or to about 50, or to about 60, or to about 70 residues. In certain embodiments the peptide ranges in length from about 2, 3, 4, 5, 7, 9, 10, or 11residues to about 60, 50, 45, 40, 45, 30, 25, 20, or 15 residues. In certain embodiments the peptide ranges in length from about 8, 9, 10, 11, or 12 residues to about 15, 20 or 25 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers.

The term “disease” can refer to an abnormal condition affecting the body of an organism. The term “disorder” can refer to a functional abnormality or disturbance.

“Alzheimer's disease” can refer to a degenerative disease most associated with dementia. It is characterized by the formation of protein aggregates that assemble into fibrillar structures. Alzheimer's Disease is associated with: 1) the formation of neuritic plaques comprising amyloid beta protein and/or neurofibrillary tangles comprising tau proteins (primarily located in the hippocampus and cerebral cortex) and, 2) an impairment in both cognitive and non-cognitive functions, for example, impairment in learning and memory, emotion, and coordination. “Alzheimer's disease” as used herein includes the different kinds of Alzheimer's disease, including but not limited to early onset family type Alzheimer's disease and late onset sporadic Alzheimer's disease.

A “polysulfide” can refer to a member of a class of chemical compounds containing one or more groups of atoms of the element sulfur linked together by covalent bonds. For example, the polysulfide can comprise Na₂S, Na₂S₂, Na₂S₃, Na₂S₄, organic sulfane sulfurs, polythionates, or any combination thereof.

A “test candidate” can refer to an experimental candidate used in a screening process to identify activity, non-activity, or other modulation of a particularized biological target or pathway. For example, the test candidate can comprise a nitric oxide (NO) or a test candidate that comprises NO.

In embodiments, the “test candidate” can comprise a drug library. A “drug library” can refer to a collection of chemicals that can be used for high-throughput screening and other processes for drug development.

“Incubating” can refer to maintaining something, such as a chemically active system, under conditions favorable for a reaction.

In embodiments, the reaction mixture can be incubated for a period. In embodiments, the reaction mixture can be incubated for about 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, or more than 12 hours. For example, the reaction mixture can be incubated for 6 hours.

In embodiments, the reaction mixture can be incubated in a reaction vessel. The term “reaction vessel” can refer to any container in which a reaction can occur in accordance with the methods described herein. For example, the reaction vessel can comprise a dish, a tube. or a multiwell plate.

As described herein, embodiments comprise in vitro methods for detecting the level of β-amyloid fragment peptide aggregation. The terms “detection” and “detecting” can be used in the context of detecting biomarkers, detecting peptides, detecting proteins, or of detecting a condition, detecting a disease or a disorder. For example, methods of detecting comprise an immunoassay (e.g., an immunoblot), mass spectrometry an aggregation assay, ultracentrifugation, size-exclusion chromatography, gel electrophoresis, or dynamic light scattering measurements.

An “immunoassay” can refer to any assay which detects, identifies, characterizes, quantifies, or otherwise measures an amino acid target in a sample. For example, an amino acid target can be a small peptide, a polypeptide, a protein, or proteinaceous macromolecule. Immunoassay's include, for example, direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays. Immunoassays use antibodies or antibody fragments, but can also use binding proteins or carrier proteins which bind target molecules with high specificity.

A “western blot” can refer to an antibody-based technique used to detect the presence, size and abundance of specific proteins within a sample. In embodiments, western blots with both denaturing and non-denaturing gels were performed. A “denaturing gel” can refer to a gel that is ran under conditions that disrupt the natural structure of DNA/RNA or protein, causing the separation of a nucleic acid duplex into two single strands. A “non-denaturing gel” can refer to a gel that is ran under conditions that no disruption of structure is introduced to analytes.

“Mass spectrometry” can refer to a sensitive and accurate technique for separating and identifying molecules. Mass spectrometry is the preferred method to detect mass-distinguishable products of the invention and thus identify and/or quantitate target nucleic acids.

In embodiments, the level of β-amyloid fragment peptide aggregation is indicative of whether the test candidate prevents or reduces β-amyloid fragment peptide aggregation. For example, the β-amyloid fragment peptide aggregation can be reduced by about 0.1%, 0.25%, 0.5%, 0.75%, 1%, 2%, 3%, 4%, 5%, 6%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Embodiments can comprise comparing the presence, absence, or level of β-amyloid fragment peptide aggregation to that of a control sample, a reference, or a threshold value.

The term “comparing” can refer to a comparison of corresponding parameters or values. For example, “comparing” can refer to comparing the level of β-amyloid fragment peptide aggregation to that of a control sample, a reference, or a threshold value.

A “control” or “control sample” can refer to a sample in which the subjects or reagents of the experiment are treated as in a parallel experiment except for omission of a procedure, reagent, or variable of the experiment. In some embodiments, the control sample is used as a standard of comparison in evaluating experimental effects. In embodiments, the control sample comprises a positive control, a negative control, or both. A “positive control” can refer to a sample in an experiment that receives a treatment with a known, expected result, and therefore can show a particular change during the experiment. A “negative control” can refer to a sample in an experiment that does not receive treatment and, therefore, does not show any change during the experiment.

A “reference” or “reference sample” can refer to a sample where a specific readout and well-known response is expected.

A “threshold value” can refer to the magnitude or intensity that must be exceeded for a certain reaction, phenomenon, result, or condition to occur or be considered relevant. The relevance can depend on context, e.g., it can refer to a positive, reactive or statistically significant relevance.

Embodiments of the invention can further comprise selecting the test candidate as a therapeutic agent if β-amyloid fragment peptide aggregation is prevented or reduced.

An “agent” can refer to any small molecule chemical compound, antibody, nucleic acid molecule, or polypeptide, or fragments thereof.

The term “therapeutic agent” can refer to any activating agent for treating disease.

Embodiments as described herein can refer to a high-throughput method for detecting β-amyloid fragment peptide aggregation. The term “high-throughput” or “high-throughput screening” can refer to a drug discovery process that allows automated testing of large numbers of chemical and/or biological compounds for a specific biological target

Aspects of the invention are also drawn to an in vitro method of identifying or monitoring β-amyloid fragment peptide aggregation in a sample. For example, the method comprises detecting β-amyloid fragment peptide aggregation in a sample comprising at least one β-amyloid fragment peptide, at least one polysulfide, and at least one test candidate; and selecting the test candidate as a therapeutic agent if β-amyloid fragment peptide aggregation is prevented or reduced.

The term “identifying” can refer to determining if an element is present or not. For example, an embodiment of the method can comprise identifying β-amyloid fragment peptide aggregation in a sample.

The term “monitoring” can refer to observing and/or checking the progress or quality of something over a period of time. For example, an embodiment of the method can comprise identifying β-amyloid fragment peptide aggregation in a sample.

Still further, aspects of the invention are drawn towards a kit for detecting β-amyloid fragment peptide aggregation in a sample. For example, an embodiment of the kit can comprise at least one β-amyloid fragment peptide, at least one polysulfide, at least one reaction vessel, a reaction media, and instructions for use. As described herein, the kit can further comprise at least one test candidate and/or at least one control. For example, the at least one test candidate can comprise a drug library. For example, the at least one control can comprise a positive control, a negative control, or both.

The instructions of use of the kits is not limited in its form. In one embodiment, the instructions of use can include information about production of the compound, molecular weight of the compound, concentration, date of expiration, batch or production site information, and so forth. In one embodiment, the instructions of use comprises methods of administering the therapeutic combination composition, e.g., in a suitable dose. dosage form, or mode of administration (e.g., a dose, dosage form, or mode of administration described herein), to treat a subject who has Alzheimer's Disease). The information can be provided in a variety of formats, include printed text, computer readable material, video recording, or audio recording, or information that provides a link or address to substantive material.

The composition in the kit can include other ingredients, such as a solvent or buffer, a stabilizer, or a preservative. The reaction mixture can be provided in any form, e.g., liquid, dried or lyophilized form, or for example, substantially pure and/or sterile. When the agents are provided in a liquid solution, the liquid solution is an aqueous solution. When the agents are provided as a dried form, reconstitution can be by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., in a given ratio. For example, the kit includes a plurality of syringes, ampules, foil packets, blister packs, or medical devices, e.g., each containing a single combination unit dose. The containers of the kits can be airtight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### The Effect of Polysulfide on β-Amyloid Protein Aggregation

### Introduction

### What is β-Amyloid?

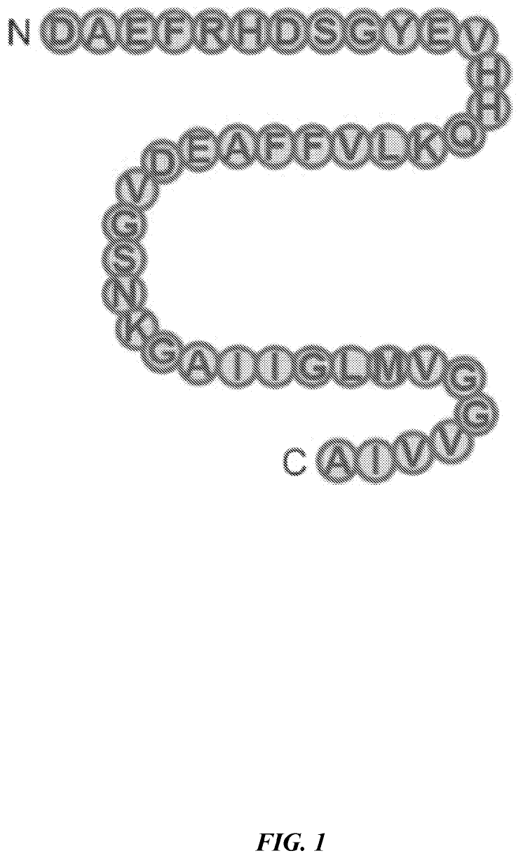

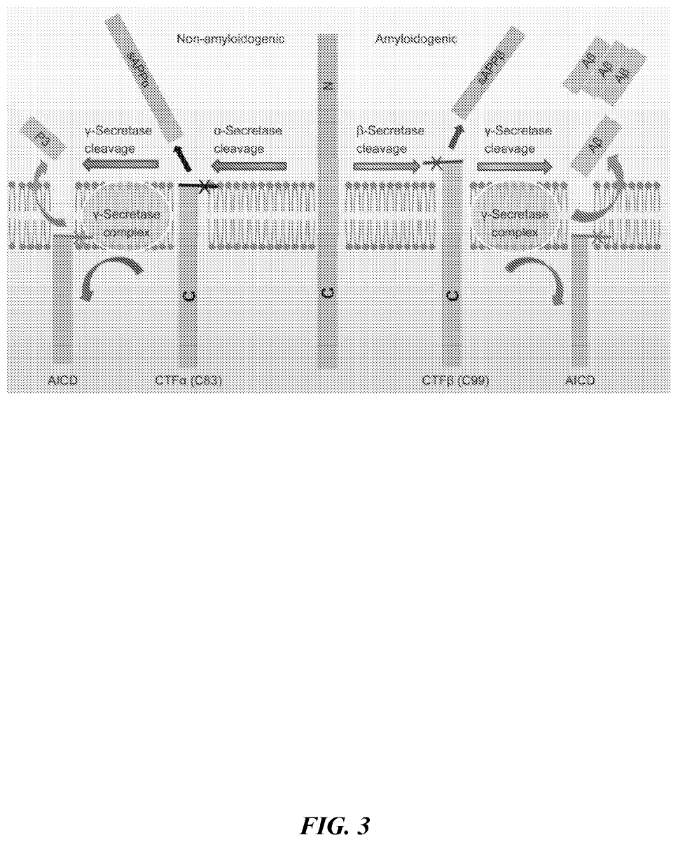

β-Amyloid is the fibrillar form of the 40-42 amino acid peptide that forms the amyloid plaques found in Alzheimer's Disease (AD) patients (FIG. 1). β-amyloid is derived from the amyloid precursor protein (APP), a membrane glycoprotein that plays a role in nervous system function (FIG. 3; adapted from Chen et al. 2017). The 1-42 and 1-40 β-Amyloid fragments are the most dominant in individuals with AD. The 25-35 fragment is known to contribute to aggregation and cytotoxicity in the nervous system (Kandel et al. 2019).

### How Does Sulfide Relate to β-Amyloid? Why is This Important?

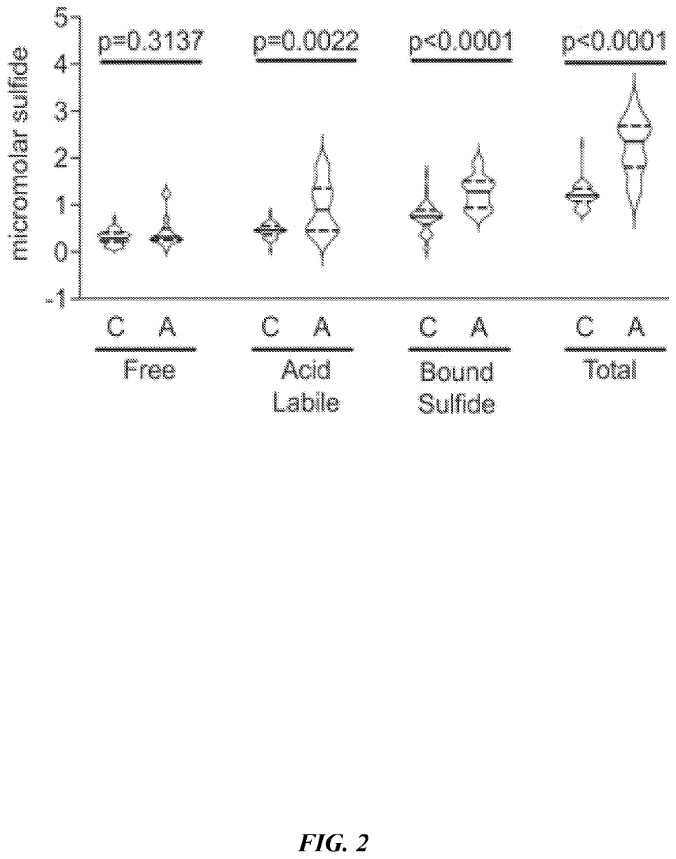

Alzheimer's Disease (AD) is characterized by the deposition of amyloid plaques between neurons in the brain (Murphy, LeVine 2010). Plasma from AD individuals have been shown to have increased levels of Total Sulfide (FIG. 2: Disbrow et al. 2021). Little is known about the relationship between β-Amyloid peptide and sulfide: sulfide's nucleophilic nature may play a role in β-Amyloid modification. Exploring the relationship between sulfide levels and β-Amyloid aggregation will lead to a better understanding of the mechanism of AD development.

### Objectives

Our data indicates that polysulfide induces modifications in β-Amyloid peptide and aggregate formation.

### Objectives

The objectives of this study include: (1) To test whether polysulfide treatment changes the expression of β-Amyloid protein using western blotting; and (2) to test how polysulfide treatment modifies the peptide structure of various fragments of β-Amyloid using mass spectrometry.

### Methods

Three β-Amyloid fragments were used: 1-42, 1-40, and 25-35 (mass spectrometry only).

Four sulfides were used: Na₂S, Na₂s₂, Na₂s₃, and Na₂S₄.

Western Blotting: To quantify expression of β-Amyloid in these experiments. western blots with both denaturing and non-denaturing gels were used. 100 μM concentrations of polysulfide donors were added to 1 μg of β-Amyloid protein and their effects were compared. Three trials were performed for the 1-42 β-amyloid fragment and one trial was performed for the 1-40 fragment.

Mass Spectrometry: To assess the effects of polysulfide treatment on the β-Amyloid amino acid sequence, mass spectrometry was used. 100 μM concentrations of polysulfide donors were added to 1 μg of β-Amyloid protein in HEPES buffer at 7.2 pH and then incubated at 37° C. for 6 hours. Three trials were performed.

### Results

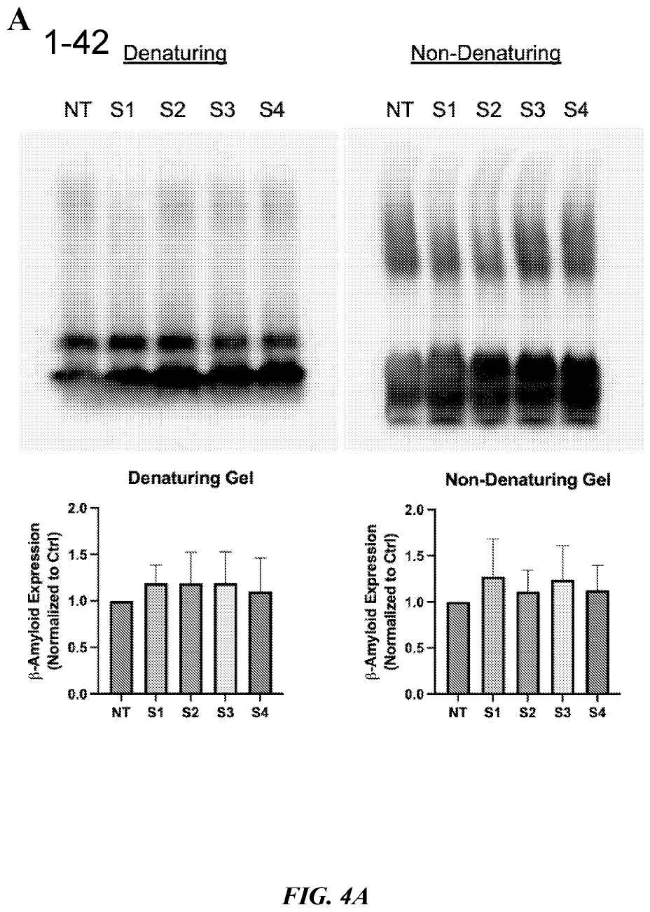

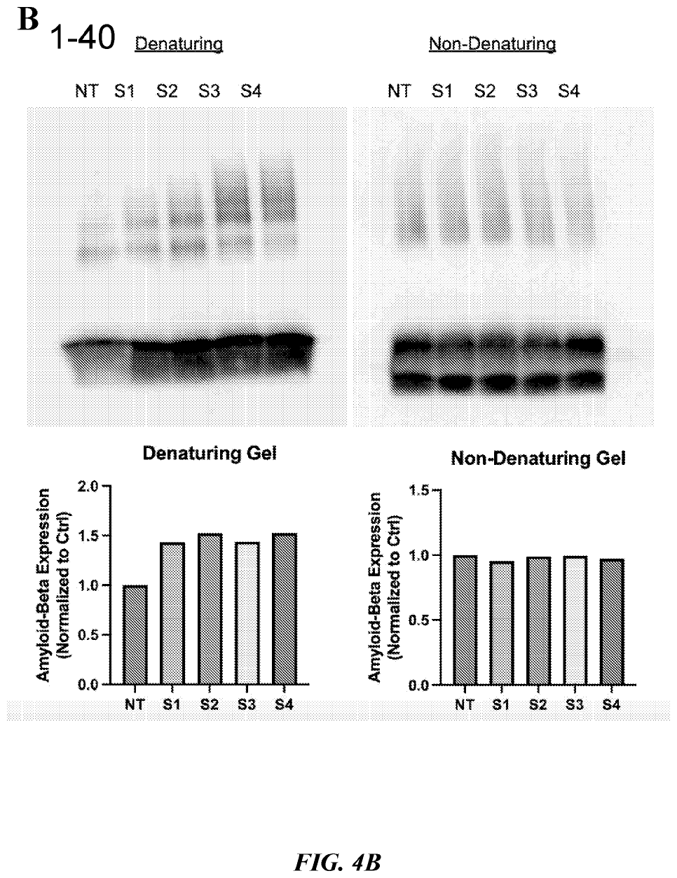

Western Blot analysis for β-Amyloid fragments 1-42 and 1-40 was performed (FIG. 4). Fragment 1-42 did not show a significant trend in β-Amyloid expression when treated with polysulfide (FIG. 4, panel A). Some trials showed the untreated group (NT) having lower β-Amyloid expression than the treated groups, which may indicate increased aggregation with polysulfide treatment. Fragment 1-40 showed increased β-Amyloid expression in the treated groups in the denaturing gel (FIG. 4, panel B). The stalk-like formation of bands in this blot indicate an increasing effect on β-Amyloid as polysulfide levels are increased.

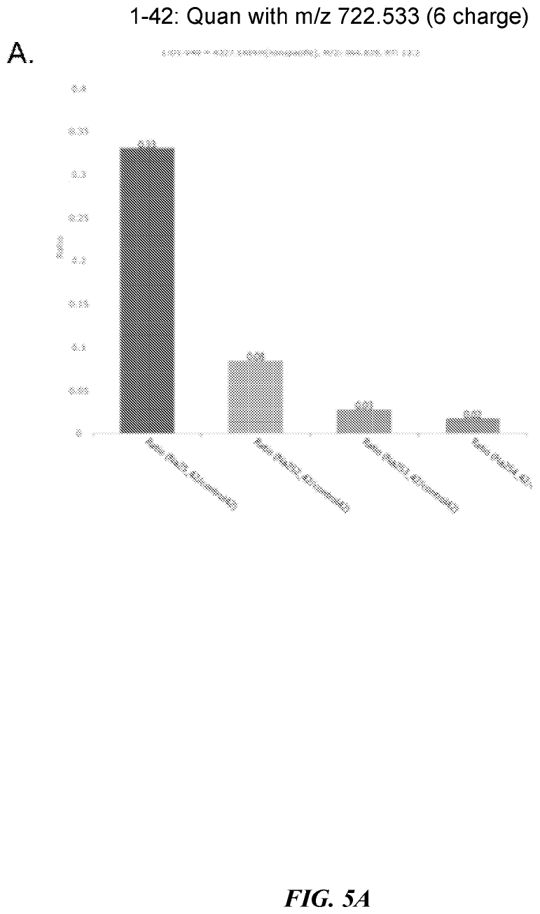

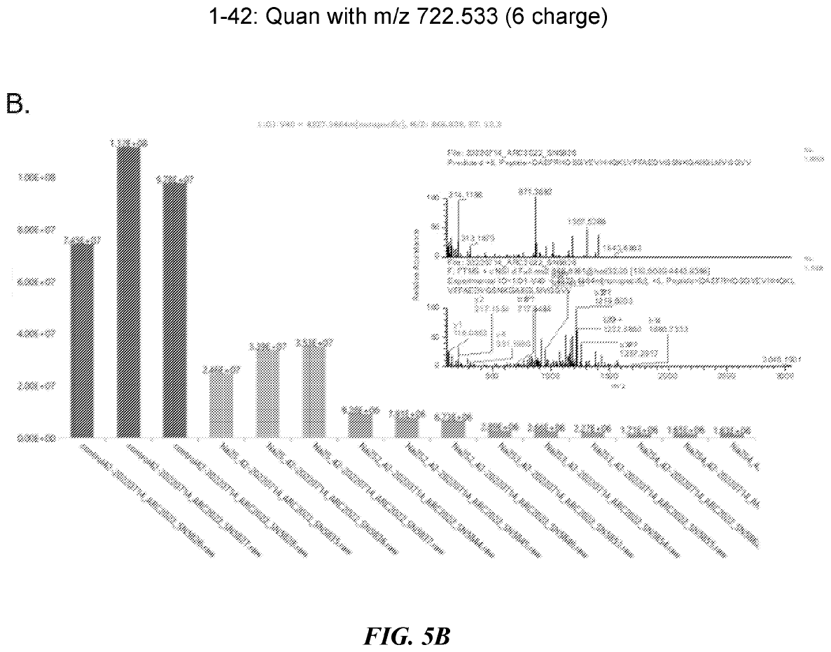

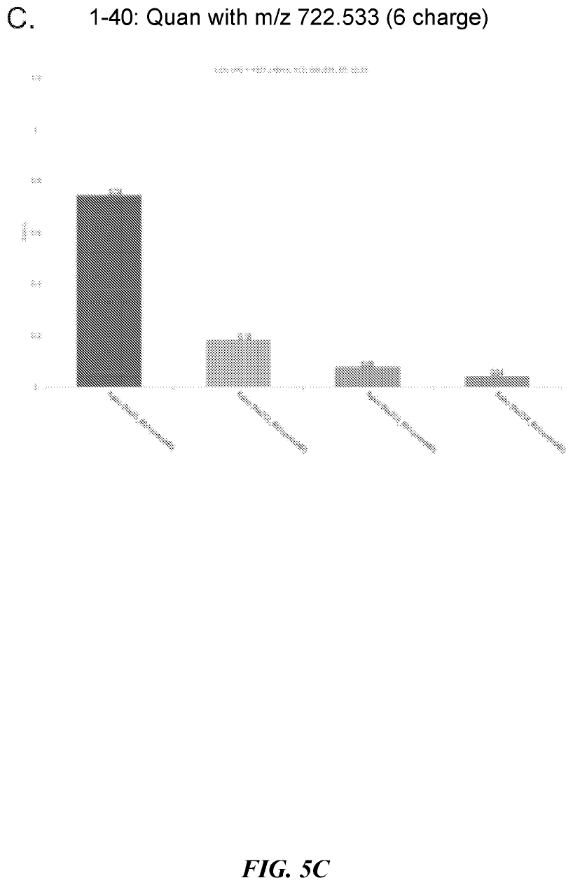

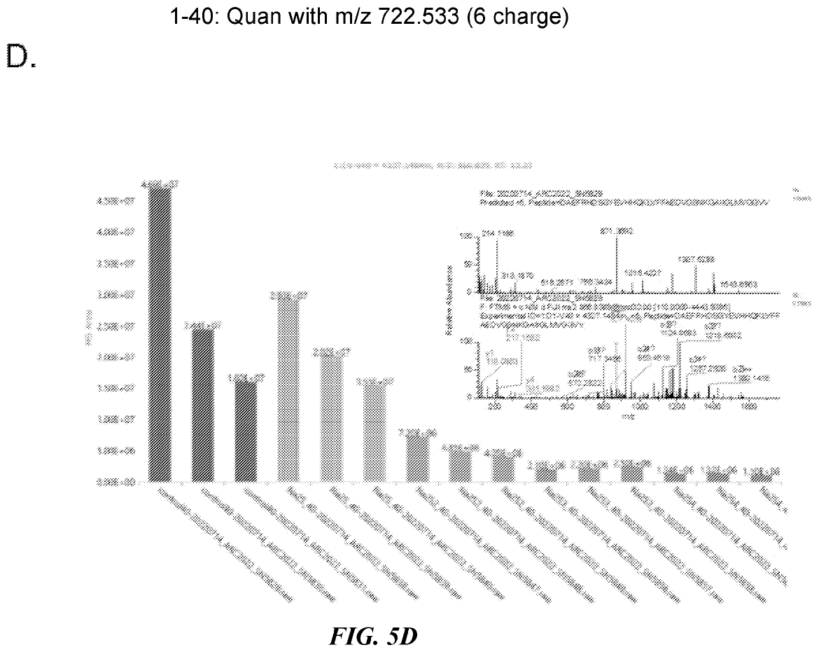

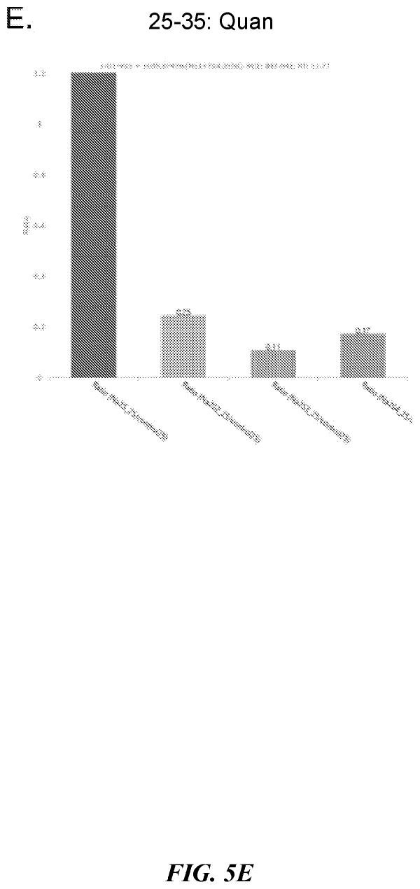

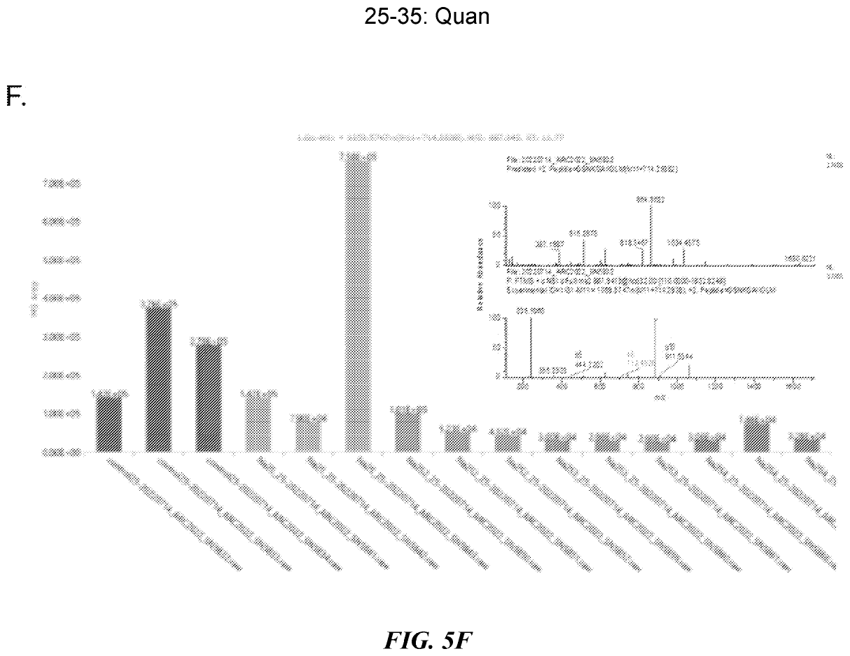

Mass spectrometry analysis was performed post-treatment with polysulfide (FIG. 5). Mass spectrometry data for β-Amyloid fragment 1-42 (FIG. 5, panels A-B), fragment 1-40 (FIG. 5, panels C-D), and fragment 25-35 (FIG. 5, panels E-F) are provided herein. Fragments 1-42 (FIG. 5, panel A) and 1-40 (FIG. 5, panel C) showed a gradual decrease in the ratio of post-treatment peptide to control peptide as the amount of sulfide treatment increased. Fragment 25-35 (FIG. 5, panel E) showed three modifications, occurring at the M35 residue: M+714.2938, M+416.1985, and M+238.0981.

### Conclusions

Results from western blotting support that β-Amyloid protein expression is affected by polysulfide treatment. Mass spectrometry data indicates that polysulfides modify the amino acid structure of β-Amyloid at the M35 residue.

Future Directions for this study include: (1) To test the effects of nitric oxide (NO) treatment on β-Amyloid as a potential countermeasure to block the effects of polysulfide; and (2) To identify β-Amyloid aggregate/precipitate formation as a result of polysulfide treatment using fluorescence spectrometry.

### Example 2

### Sulfane Sulfur Modification and Aggregation of Beta Amyloid Peptide

This example describes that sulfane sulfur chemicals, such as persulfide and polysulfides, chemically modify beta amyloid peptides (1-42, 1-40, and 25-35) at methionine amino acid and increases peptide aggregation. These events are pathologically relevant to Alzheimer's Disease (AD) as these patients have been reported to have increased plasma bound sulfane sulfur levels and increased beta amyloid plaque formation. In addition, increasing nitric oxide levels through exogenous donors or endogenous molecular pathways may antagonize sulfane sulfur mediated beta amyloid protein modification and AD patients also show reduced plasma nitric oxide metabolite levels.

Products useful for the embodiments described herein include without limitation diagnostic tests, targets for therapeutic intervention, and commercial source of synthetic toxic beta amyloid for research purposes.

Competitive Advantages include, for example: 1) embodiments herein provide new therapeutic targets for AD interventions; 2) embodiments herein provide a new way to evaluate or monitor beta amyloid plaque formation; and 3) embodiments herein provide a unique tool for studying beta amyloid aggregate/fibril cellular toxicity.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. An in vitro drug-screening method, wherein the method comprises incubating, for a period of time, a reaction mixture comprising at least one β-amyloid fragment peptide, at least one polysulfide, and at least one test candidate, and detecting the presence or absence of β-amyloid fragment peptide aggregation, wherein the presence or absence of β-amyloid fragment peptide aggregation is indicative of whether the test candidate prevents or reduces β-amyloid fragment peptide aggregation.

2. The in vitro drug-screening method of claim 1, wherein the β-amyloid fragment peptide comprises 1-40, 1-42, 25-35, or any combination thereof.

3. The in vitro drug-screening method of claim 1, wherein the polysulfide comprises Na₂S, Na₂S₂, Na₂S₃, Na₂S₄, organic sulfane sulfurs, polythionates, or any combination thereof.

4. The in vitro drug-screening method of claim 1, wherein detecting comprises an immunoassay, mass spectrometry, an aggregation assay, ultracentrifugation, size-exclusion chromatography, gel electrophoresis, or dynamic light scattering measurements.

5. The in vitro drug-screening method of claim 4, wherein the immunoassay is an immunoblot.

6. The in vitro drug-screening method of claim 1, wherein the presence or absence of β-amyloid fragment peptide aggregation is compared to that of a control sample.

7. The in vitro drug-screening method of claim 6, wherein the control sample comprises a positive control, a negative control, or both.

8. The in vitro drug-screening method of claim 1, further comprising selecting the test candidate as a therapeutic agent if β-amyloid fragment peptide aggregation is prevented or reduced.

9. The in vitro drug-screening method of claim 1, wherein the reaction mixture is incubated in a reaction vessel.

10. The in vitro drug-screening method of claim 9, wherein the reaction vessel comprises a dish, a tube, or a multiwell plate.

11. The in vitro drug-screening method of claim 1, wherein the at least one test candidate comprises a drug library.

12. The in vitro drug-screening method of claim 1, wherein the at least one test candidate comprises a nitric oxide (NO) or a test candidate that comprises NO.

13. The in vitro drug-screening method of claim 1, wherein the method is a high-throughput method.

14. An in vitro method of identifying or monitoring ß-amyloid fragment peptide aggregation in a sample, comprising
detecting β-amyloid fragment peptide aggregation in a sample comprising at least one β-amyloid fragment peptide, at least one polysulfide, and at least one test candidate;
and selecting the test candidate as a therapeutic agent if β-amyloid fragment peptide aggregation is prevented or reduced.

15. The in vitro method of claim 14, wherein the β-amyloid fragment peptide comprises 1-40, 1-42, 25-35, or any combination thereof.

16. The in vitro method of claim 14, wherein the polysulfide comprises Na₂S, Na₂S₂, Na₂S₃, Na₂S₄, organic sulfane sulfurs, polythionates, or any combination thereof.

17. The in vitro method of claim 14, wherein detecting comprises an immunoassay, mass spectrometry, an aggregation assay, ultracentrifugation, size-exclusion chromatography, gel electrophoresis, or dynamic light scattering measurements.

18. The in vitro method of claim 17, wherein the immunoassay is an immunoblot.

19. The in vitro method of claim 14, wherein the level of β-amyloid fragment peptide aggregation is compared to that of a control sample.

20. The in vitro method of claim 19, wherein the control sample comprises a positive control, a negative control, or both.

21. The in vitro method of claim 14, wherein the β-amyloid fragment peptide, at least one polysulfide, and at least one test candidate are co-incubated for a period of time in a reaction vessel.

22. The in vitro method of claim 21, wherein the reaction vessel comprises a dish, a tube, or a multiwell plate.

23. The in vitro method of claim 14, wherein the at least one test candidate comprises a drug library.

24. The in vitro method of claim 14, wherein the at least one test candidate comprises a nitric oxide (NO) or a test candidate that comprises NO.

25. The in vitro method of claim 14, wherein the method is a high-throughput method.

26. A kit comprising at least one β-amyloid fragment peptide, at least one polysulfide, at least one reaction vessel, a reaction media, and instructions for use.

27. The kit of claim 26, further comprising at least one test candidate.

28. The kit of claim 27, where in the at least one test candidate comprises a drug library.

29. The kit of claim 26. further comprising at least one control.

30. The kit of claim 29, wherein the at least one control comprises a positive control, a negative control, or both.
